(19) United States
(12) Patent Application Publication
(10) Pub. No.: US 2025/0332389 A1
(43) Pub. Date: Oct. 30, 2025

(54) PERFUSION BALLOON CATHETERS WITH THERAPEUTIC COATINGS

(71) Applicant: Boston Scientific Scimed, Inc., Maple Grove, MN (US)

(72) Inventors: Javier Palomar-Moreno, Galway (IE); Michelle Hannon, Galway (IE); Jeffrey Madden, Galway (IE); John Kilcooley, Gort (IE)

(73) Assignee: Boston Scientific Scimed, Inc., Maple Grove, MN (US)

(21) Appl. No.: 19/189,414

(22) Filed: Apr. 25, 2025

Related U.S. Application Data
(60) Provisional application No. 63/638,500, filed on Apr. 25, 2024.

(51) Int. Cl. A61M 25/10; A61M 25/00

(52) U.S. Cl. CPC A61M 25/1002; A61M 25/0043; A61M 2025/0059; A61M 2025/1004; A61M 2025/105; A61M 2025/1075; A61M 2202/0007; A61M 2205/0238; A61M 2205/3334; A61M 2210/125

(57) ABSTRACT

A perfusion catheter for drug delivery to tissue, the perfusion catheter comprising: an elongate catheter shaft having a proximal end region and a distal end region and including an inflation lumen extending between the proximal end region and the distal end region; a balloon positioned adjacent to the distal end region of the elongate catheter shaft and in fluid communication with the inflation lumen, the balloon being configured to move between an collapsed configuration and an expanded configuration where a first region of a surface of the balloon defines a perfusion channel; and a therapeutic coating disposed on a second region of the surface of the balloon, wherein the second region is configured to contact the tissue in the expanded configuration.

### CROSS-REFERENCE TO RELATED APPLICATIONS

This application claims the benefit of priority under 35 U.S.C. § 119 of U.S. Provisional Application No. 63/638,500, filed Apr. 25, 2024, the entire disclosure of which is hereby incorporated by reference.

### TECHNICAL FIELD

The disclosure pertains to medical devices and more particularly to perfusion balloon catheters with therapeutic coatings for drug delivery such as drug delivery to cardiac tissue.

### BACKGROUND

A wide variety of medical devices have been developed for medical use, for example, intravascular use. Some of these devices include guidewires, catheters, balloons, stents, and the like. These devices are manufactured by any one of a variety of different manufacturing methods and may be used according to any one of a variety of methods. Some of these medical devices may include a therapeutic agent. Of the known medical devices and methods, each has certain advantages and disadvantages. There is an ongoing need to provide alternative medical devices as well as alternative methods for manufacturing and using medical devices.

### SUMMARY

The present disclosure pertains to medical devices and more particularly to perfusion balloon catheters with therapeutic coatings for drug delivery to cardiac tissue.

In an example, a perfusion catheter for drug delivery to tissue is provided. The perfusion catheter comprising: an elongate catheter shaft having a proximal end region and a distal end region and including an inflation lumen extending between the proximal end region and the distal end region; a balloon positioned adjacent to the distal end region of the elongate catheter shaft and in fluid communication with the inflation lumen, the balloon being configured to move between a collapsed configuration and an expanded configuration where a first region of a surface of the balloon defines a perfusion channel; and a therapeutic coating disposed on a second region of the surface of the balloon, wherein the second region is configured to contact the tissue in the expanded configuration.

Alternatively or additionally to any of the examples above, in another example, the first region is free of the therapeutic coating.

Alternatively or additionally to any of the examples above, in another example, the perfusion channel is configured to permit blood to flow substantially longitudinally about the balloon, and wherein a flow rate of the blood is greater than about 20 milliliters per minute.

Alternatively or additionally to any of the examples above, in another example, the perfusion channel has a width in a range from about 2 microns (0.0000787 inches) to about 50 microns (0.00197 inches) and a height in a range from about 2 microns (0.0000787 inches) to about 50 microns (0.00197 inches).

Alternatively or additionally to any of the examples above, in another example, the perfusion channel is formed of a plurality of perfusion channels.

Alternatively or additionally to any of the examples above, in another example, each of the plurality of perfusion channels is substantially the same shape, substantially the same size, or both.

Alternatively or additionally to any of the examples above, in another example, the second region is configured to be a first distance from a longitudinal axis of the perfusion catheter in the collapsed configuration, a second distance from the longitudinal axis of the perfusion catheter in the expanded configuration, and wherein the second distance is greater than the first distance.

Alternatively or additionally to any of the examples above, in another example, the perfusion channel is an individual perfusion channel.

Alternatively or additionally to any of the examples above, in another example, the balloon is a toroidal shaped balloon, and wherein the individual perfusion channel is a lumen extending through the toroidal shaped balloon.

Alternatively or additionally to any of the examples above, in another example, the perfusion catheter comprises struts extending radially from the elongate catheter shaft and being in fluid communication with the toroidal shaped balloon and an inflation lumen in the elongate catheter shaft.

Alternatively or additionally to any of the examples above, in another example, the second region is configured to be recessed a distance from the tissue when the balloon is in the collapsed configuration.

Alternatively or additionally to any of the examples above, in another example, the perfusion channel further comprises a substantially longitudinally extending perfusion channel that is configured to permit perfusion of blood from a first side of the balloon to a second side of the balloon when the balloon is in the expanded configuration.

Alternatively or additionally to any of the examples above, in another example, the perfusion channel comprises a gap between the first region of the surface of the balloon and the tissue.

Alternatively or additionally to any of the examples above, in another example, the perfusion channel is formed at least in part by an elongate perfusion tube coupled to and extending longitudinally along the surface of the balloon.

Alternatively or additionally to any of the examples above, in another example, the therapeutic coating comprises a plurality of everolimus crystals.

In another example, a perfusion catheter for drug delivery to cardiac tissue is provided. The perfusion catheter comprising: an elongate catheter shaft having a proximal end region and a distal end region and including a guidewire lumen and an inflation lumen extending between the proximal end region and the distal end region; a balloon positioned adjacent to the distal end region of the elongate catheter shaft and in fluid communication with the inflation lumen, the balloon being configured to move between a collapsed configuration and an expanded configuration where a first region of an exterior surface of the balloon is uncoated and defines a perfusion channel configured to permit substantially longitudinal blood flow about the balloon; and a therapeutic coating disposed on a second region of the exterior surface of the balloon, wherein the second region is configured to contact the cardiac tissue in the expanded configuration.

Alternatively or additionally to any of the examples above, in another example, the first region is included in a plurality of first regions, wherein the second region is included in a plurality of second regions.

Alternatively or additionally to any of the examples above, in another example, the plurality of first regions and the plurality of second regions are configured to alternate about an abluminal surface of the balloon.

In another example a perfusion catheter for drug delivery to cardiac tissue is provided. The perfusion catheter comprising: an elongate catheter shaft having a proximal end region and a distal end region and including an inflation lumen extending between the proximal end region and the distal end region; a balloon positioned adjacent to the distal end region of the elongate catheter shaft and in fluid communication with the inflation lumen, the balloon being configured to move between an collapsed configuration and a radially expanded configuration where a plurality of first regions of an exterior surface of the balloon are uncoated and define a plurality of perfusion channels extending substantially longitudinally about the exterior surface of the balloon and being configured to permit blood to flow substantially longitudinally about the exterior surface, wherein a sum of the respective flow rates of the blood through each of the plurality of perfusion channels is greater than about 20 milliliters per minute; and a therapeutic coating disposed on a plurality of second regions of the exterior surface of the balloon, wherein the plurality of second regions are configured to contact the cardiac tissue in the radially expanded configuration.

Alternatively or additionally to any of the examples above, in another example, the plurality of first regions each have a substantially concave shape relative to a longitudinal axis of the perfusion catheter when the balloon is in the collapsed configuration; and the plurality of second regions each have a substantially convex shape relative to the longitudinal axis of the perfusion catheter when the balloon is in the radially expanded configuration.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic side view of an example drug delivery balloon catheter.

FIG. 2A is a cross-sectional view taken through line 2-2 in FIG. 1 when the balloon is in a collapsed configuration.

FIG. 2B is a cross-sectional view taken through line 2-2 in FIG. 1 when the balloon is in an expanded configuration.

FIG. 3 depicts an example drug-coated balloon in an expanded configuration in contact with a vessel wall.

FIG. 4A illustrates another example drug-coated balloon in a collapsed configuration.

FIG. 4B illustrates the balloon of FIG. 4B in an expanded configuration.

FIG. 4C is a cross-sectional view taken through line 3-3 of the balloon of FIG. 4A.

FIG. 4D is a cross-sectional view taken through line 3-3 of the balloon of FIG. 4B.

FIG. 5A illustrates an example drug-coated balloon.

FIG. 5B illustrates a cross-sectional view taken through line 5-5 of the balloon of FIG. 5A in a collapsed configuration.

FIG. 5C illustrates a cross-sectional view taken through line 5-5 of the balloon of FIG. 5A in a partially expanded configuration.

FIG. 5D illustrates a cross-sectional view taken through line 5-5 of the balloon of FIG. 5A in an expanded configuration.

FIG. 6A illustrates an example drug-coated toroidal balloon in an expanded configuration.

FIG. 6B illustrates another view of the toroidal balloon of FIG. 6A.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Drug coated medical devices such as drug coated stents, drug coated balloons, and the like may be used to treat small vessel occlusions and/or vascular disease. For instance, a drug coated balloon may include a drug or other therapeutic agent applied to an exterior surface that may be exposed or unexposed when the balloon is in a collapsed/deflated configuration. Portions of the, or an entire drug-coated exterior surface may contact a vessel wall when the balloon is expanded (e.g., inflated). For instance, the drug-coated exterior surface may typically have a circular cross-section or otherwise may be configured to contact an entire luminal surface of the vessel adjacent to the balloon. As such, these approaches may entirely or at least substantially restrict perfusion about the balloon when expanded and in contact with the vessel wall. As such, the existing drug coated balloons may not be suitable for patients with various conditions, such as those with coronary artery disease (CAD). A perfusion catheter has an indication to prevent ischemia when treating coronary artery disease (CAD). A prolonged balloon inflation may be required for drug delivery to tissue, particularly for drugs with low lipophilicity and slow tissue absorption. Such a prolonged inflation can trigger ischemia and decompensate the ventricular function of fragile patients with low ejection fraction and cardiac insufficiency, causing procedural complications. For example, it has been determined that blocking coronary artery blood flow (ceasing or substantially ceasing perfusion) to cardiac tissue even for a relatively short period of time (e.g., 30 to 60 seconds) while a drug-delivery balloon is in an expanded configuration in contact with a vessel can accelerate disease (e.g., CA D) progression due at least the resultant lack of perfusion about the expanded balloon and/or otherwise cause complications associated with drug delivery via the drug delivery balloon, particularly in CA D patients (e.g., patients with cardiac insufficiency or poor ventricular ejection fraction where a prolonged balloon inflation can trigger ischemia and decompensate the left ventricular function).

As such, the disclosure is directed to perfusion catheters for drug delivery to tissue (e.g., cardiac tissue). The perfusion catheters employ a drug-coated balloon that is configured to permit blood flow substantially longitudinally about the drug-coated balloon when the balloon is in an expanded configuration in contact with a vessel wall (e.g., to deliver a drug in a therapeutic coating on a surface of the balloon to the vessel wall). Namely, the drug-coated perfusion balloons herein can define at least one perfusion channel (e.g., a substantially longitudinally extending perfusion channel) configured to permit blood to flow substantially longitudinally about the expanded balloon. Stated differently, blood can flow from a distal or proximal end of the expanded balloon via a perfusion channel to the other of the distal or proximal end of the expanded balloon when the expanded balloon is in contact with tissue of a vessel wall. Thus, unlike the previous drug coated balloons which completely or substantially cease perfusion (e.g., less than twenty millimeters of blood per minute) about the expanded drug delivery balloon, the perfusion catheters herein permit a sufficient amount of blood to flow substantially longitudinally about the balloon when the balloon is in an expanded configuration. For example, the blood flow rate via the perfusion channel can be greater than about twenty milliliters per minute. That is, the perfusion catheters herein can maintain sufficient perfusion (e.g., greater than about twenty milliliters per minute) during an entire duration while a balloon is in an expanded configuration and thereby can mitigate disease (e.g., CA D) progression and/or other complications typically associated with drug delivery via the expanded drug delivery balloons, particularly for CAD patients. There are other pathologies (in addition to CAD) and adjacencies that can benefit from the disclosed perfusion catheters. For instance, treating blockages in the carotid arteries that supply blood to the brain.

In some embodiments the perfusion catheters herein can provide enhanced (e.g., more accurate) drug delivery due to the balloons being configured to protect the therapeutic coating (e.g., minimize mechanical or friction loss of the therapeutic coating, which may cause drug losses to the systemic blood circulation while tracking the device and therefore delivering a diminished drug dose to the treated vessel when the balloon is inflated) on the exterior surface of the balloon during insertion of the drug deliver balloon in a vessel and/or can permit longer drug delivery time window (e.g., greater than 60 seconds, greater than two minutes, greater than three minutes, greater than four minutes, less than five minutes, etc.) due to maintaining a sufficient degree of perfusion while in an expanded configuration, as compared to existing drug coated balloons. Longer transfer times may be required for particular active pharmaceutical ingredients, drugs and/or biotherapeutics with low lipophilicity which may require longer transfer times e.g., to cross the cell membrane of treated tissue.

In some embodiments, the therapeutic coating on the surface of the balloon can include an excipient, an active agent and/or drug (e.g., an amorphous form of a drug or a crystalline form of a drug). That is, disclosed herein are medical devices with such a coating applied thereto, methods for coating, etc. Some specific beneficial agents include anti-thrombotic agents, antiproliferative agents, anti-inflammatory agents, anti-migratory agents, pro-endothelization agents and/or other agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

More specific drugs or therapeutic agents include paclitaxel, rapamycin, sirolimus, everolimus, tacrolimus, heparin, diclofenac, aspirin, Epo D, dexamethasone, estradiol, halofuginone, cilostazol, geldanamycin, ABT-578 (Abbott Laboratories), trapidil, liprostin, Actinomycin D, Resten-NG, Ap-17, abciximab, clopidogrel, Ridogrel, beta-blockers, bARK ct inhibitors, phospholamban inhibitors, and SERCA 2 gene/protein, resiquimod, imiquimod (as well as other imidazoquinoline immune response modifiers), human apolipoproteins (e.g., AI, AII, AIII, AIV, AV, etc.), vascular endothelial growth factors (e.g., VEGF-2), as well as derivatives of the forgoing, among many others.

In some embodiments, the drug may be a macrolide immunosuppressive (limus) drug. In some embodiments, the macrolide immunosuppressive drug is rapamycin, biolimus (biolimus A 9), 40-O-(2-Hydroxyethyl) rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4 (S)-yl)-prop-2′-en-1′-yl]-rapamycin, (2′: E,4'S)-40-O-(4′,5′-Dihydroxypent-2′-en-1′-yl)-rapamycin, 40-O-(2-Hydroxy) ethoxycar-bonylmethyl-rapamycin, 40-O-(3-Hydroxy) propyl-rapamycin, 40-O-(6-Hydroxy) hexyl-rapamycin, 40-O-[2-(2-Hydroxy) ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-A cetoxy)ethyl-rapamycin, 40-O-(2-Nicotinoyloxy)ethyl-rapamycin, 40-O-[2-(N-Morpholino) acetoxy]ethyl-rapamycin, 40-O-(2-N-Imidazolylacetoxy)ethyl-rapamycin, 40-O-[2-(N-Methyl-N′-piperazinyl) acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-A minoethyl)-rapamycin, 40-O-(2-A cetaminoethyl)-rapamycin, 40-O-(2-Nicotinamidoethyl)-rapamycin, 40-O-(2-(N-M ethyl-imidazo-2′-ylcarbethoxamido)ethyl)-rapamycin, 40-O-(2-Ethoxycarbonylaminoethyl)-rapamycin, 40-O-(2-T olylsulfonamidoethyl)-rapamycin, 40-O-[2-(4′,5′-Dicarboethoxy-1′,2′, 3′-triazol-1′-yl)-ethyl]-rapamycin, 42-Epi-(tetrazolyl) rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), or derivative, isomer, racemate, diastereoisomer, prodrug, hydrate, ester, or analog thereof. Other drugs may include anti-inflammatory agents such as dexamethasone, prednisolone, corticosterone, budesonide, estrogen, sulfasalazine, mesalamine, and analogues thereof; antineoplastic/antiproliferative/anti-miotic agents such as paclitaxel, 5-fluorouracil, cisplatin, vinblastine, vincristine, epothilones, endostatin, angiostatin, thymidine kinase inhibitors, and analogues thereof; anesthetic agents such as lidocaine, bupivacaine, ropivacaine, and analogues thereof; anti-coagulants; and growth factors.

In some cases, everolimus may be the drug used. Everolimus, which is also known as 40-O-(2-Hydroxyethyl) rapamycin, has the following chemical structure:

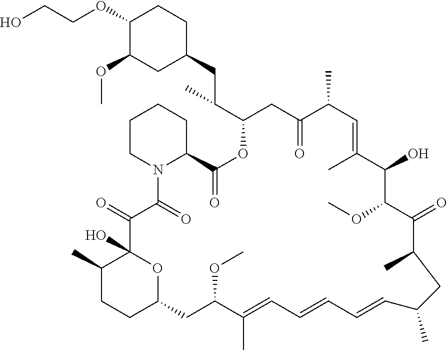 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

In some instances, providing a drug coated medical device with a drug coating that is adapted to permit an extended release profile may be beneficial in treating small vessel occlusions and/or vascular disease. In some instances, improved results may be achieved in cases where the extended release profile means that a useful fraction of the drug remains for an extended period of time, thereby increasing the efficacy of the drug in treating whatever condition is being treated, at least in part because a useful fraction of the drug remains for a longer period of time. In some instances, a drug coating with an extended release profile may mean that not as much drug is required in the coating in order to achieve a desired effect, for example.

In some instances, a therapeutic coating including encapsulated everolimus crystals may provide a 30 day tissue everolimus concentration of at least 0.5 nanograms per milligram, of at least 0.6 nanograms per milligram, of at least 0.7 nanograms per milligram, of at least 0.8 nanograms per milligram, of at least 0.9 nanograms per milligram, or may provide a 30 day tissue everolimus concentration of at least 1 nanogram per milligram, among other possibilities.

In some instances, the drug coating may include individual drug particles that are encapsulated with one or more excipients. The drug particles may include crystals of the drug, for example. Drug crystals may be formed in a variety of ways, for example. In some cases, a drug or other therapeutic agent may be available in an amorphous form, and a variety of processes may be used to convert an amorphous drug or other therapeutic agent into a crystalline drug or other therapeutic agent.

A medical device such as the balloons may be coated with a therapeutic coating (e.g., therapeutic coating composition). As an example, the therapeutic coating composition may include everolimus. Everolimus crystals may be coated with a mixture of excipients in order to form encapsulated everolimus crystals that are suspended in a coating composition and/or to bind the biotherapeutic agent to the balloon surface with a weak bond that can be broken during the balloon inflation and interaction with the vessel wall. In some instances, the medical device may be contacted with the coating composition in order to form a coating on the medical device. In some instances, the medical device or a portion thereof may be dipped into the coating composition. In some cases, vapor deposition may be used to transfer the coating composition to the medical device. In some cases, a roller coating process may be used to transfer the coating composition/formulation to the medical device. These are just examples. In some cases, the coating composition may be sprayed onto the medical device, or may be sprayed onto a particular portion or region of the medical device.

When the medical device includes an inflatable balloon, for example, the coating composition/formulation may be sprayed onto at least a portion of outer surface of the inflatable balloon in order to be able to subsequently transfer at least a portion of the drug coating to blood vessel walls. Alternative coating processes may be used such as jet dot printing, dip coating, roller coating, spray coating, vapor deposition, and/or the like, and/or other suitable coating processes. There may be little or no benefit to applying the coating composition/formulation to other portions of the medical device such as a balloon catheter shaft because the balloon catheter shaft may make incidental contact at best with the blood vessel walls, for example.

In some instances, one or more excipients may be employed. For instance, the mixture of excipients may include two or more different excipients. An example excipient may include ethyl cellulose (EC), which is a derivative of cellulose in which some of the hydroxyl groups on the repeating glucose units are converted into ethyl ether groups. The relative number of ethyl ether groups can vary depending on the particular manufacturer. EC has the following chemical structure:

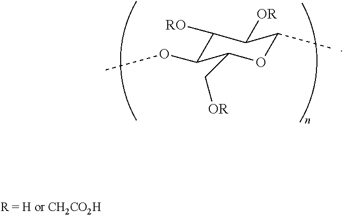

Another example excipient may include acetyl tri-butyl citrate (ATBC), which in some cases may be referred to by its IU PAC name of tributyl 2-acetyloxypropane-1,2,3-tricarboxylate. ATBC has the following chemical structure:

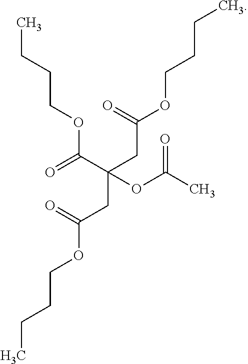 [SMILES: CCCCOC(=O)CC(CC(=O)OCCCC)(OC(C)=O)C(=O)OCCCC]

In some cases, the mixture of excipients may optionally include one or more additional excipients. In some instances, the mixture of excipients may include only EC and/or ATBC. In some instances, for example, the mixture of excipients may include two parts EC to one to six parts ATBC. As an example, coating everolimus crystals with a mixture of excipients to form encapsulated everolimus crystals suspended in a coating composition may include suspending everolimus crystals in a first solution that includes ATBC. A second solution including EC may be added to the first solution. When the second solution including EC is added to the first solution that includes the suspended everolimus crystals and ATBC, the EC mixes with the ATBC and coats the everolimus crystals. This forms a coating composition that includes encapsulated everolimus crystals held within a suspension. In some examples, individual everolimus crystals may have a coating that is less than one micron thick.

In some instances, the encapsulated everolimus crystals may be considered as including from 0 to 30 weight percent ATBC, from 0 to 30 weight percent EC and from 70 to 100 weight percent everolimus or including from 5 to 20 weight percent ATBC, from 5 to 20 weight percent EC and from 60 to 90 weight percent everolimus, among other possible values. In some instances, the encapsulated everolimus crystals may include about 85 weight percent everolimus and about 15 weight percent excipient, with the excipient being a combination of ATBC and EC. As an example, the excipient may be more than half ATBC and less than half EC. As an example, the excipient may include two parts ATBC and one part EC.

In some instances, the first solution and/or second solution may include a singular solvent or two or more solvents. In at least some instances, the solvent used for forming the first and/or second solution may include alcohols such as methanol, ethanol (EtOH), isopropanol (IPA), n-butanol, isobutyl alcohol or t-butyl alcohol; acetonitrile (ACN); ethers such as tetrahydrofuran (THF), isopropyl ether (IPE), diethyl ether (DEE); ketone solvents such as acetone, 2-butanone (MEK), or methyl isobutyl ketone (MIBK); halogenated solvents such as dichloromethane (DCM), monofluorobenzene (MFB), α,α,α-trifluorotoluene (TFT), nitromethane (NM), ethyl trifluoroacetate (ETFA); aliphatic or alicyclic hydrocarbons such as hexane, heptane, cyclohexane or the like; aromatic hydrocarbons, such as toluene or xylenes; and ester solvents such as ethyl acetate. Mixed solvents such as ethyl acetate/heptane, acetone/water, IPA/water, IPA/THF, methanol/water, IPA/heptane, or THF/heptane can also be used, for example. Other solvent systems are also contemplated. In some cases, the solvent used for forming the first solution may include cyclohexane. In some cases, the solvent used for forming the second solution may include ethyl acetate. Mixed solvents such as ethyl acetate/heptane, acetone/water, IPA/water, methanol/water, IPA/heptane, or THF/heptane can also be used, for example. Other solvent systems are also contemplated

A medical device may be adapted to be placed within a location with a vasculature. In some cases, a medical device may be adapted to be placed within an artery or a vein, for example. The medical device may include a surface that is adapted to be placed in contact with a vessel wall within the vasculature. The medical device may include a therapeutic coating that is disposed on the surface, the therapeutic coating including a plurality of everolimus crystals that are encapsulated within a coating.

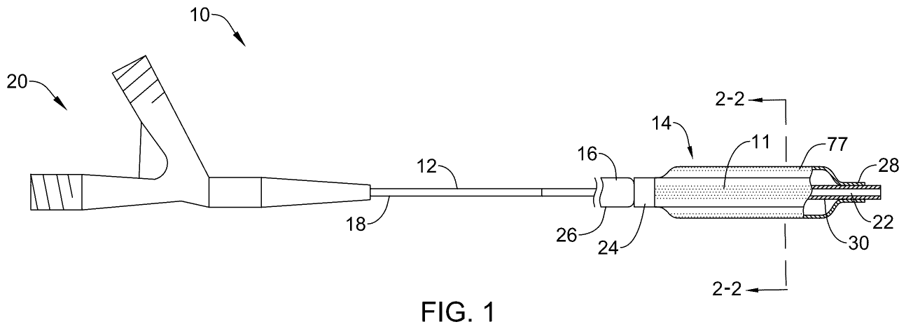

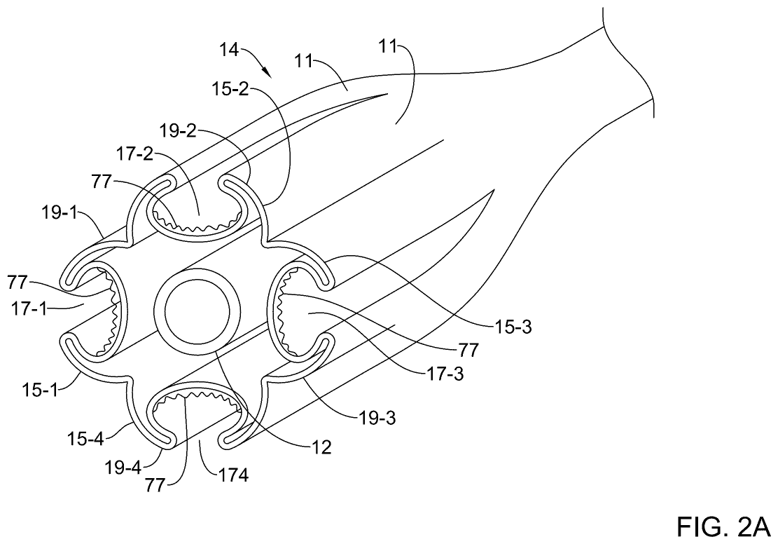

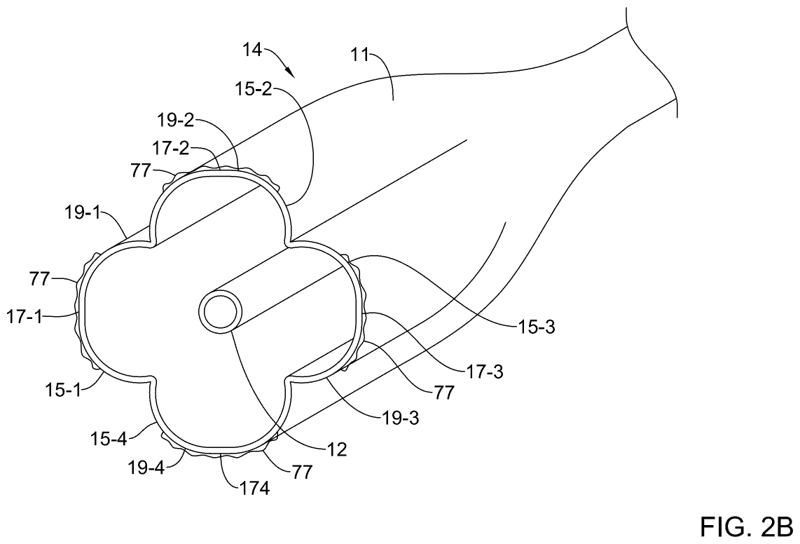

Some example drug coated medical devices, and portions thereof, are shown in FIGS. 1, 2A-2B, 3, 4A-4D, 5A-5D, and 6A-6B. For example, FIG. 1 is a schematic side view of a drug delivery balloon over-the-wire (“Y” manifold mounted on proximal end) catheter 10. A cross-sectional view (along section line 2-2) of the drug delivery balloon catheter 10 is shown in FIGS. 2A-2B. FIG. 2A is a cross-sectional view taken through line 2-2 in FIG. 1 when the balloon is in a collapsed configuration, with the drug reservoir occluded to avoid drug losses when tracking the catheter through vasculature. FIG. 2B is a cross-sectional view taken through line 2-2 in FIG. 1 when the balloon is in an expanded configuration, with the drug reservoir exposed and apposed to the vessel wall for drug transfer. In the illustrated embodiment, the catheter 10, along with other components, may include an elongated shaft 12, an inflatable balloon 14 coupled at or to a distal portion 16 of the shaft 12. The elongated shaft 12 may include a tubular member having a proximal end region or proximal portion 18, and one or more lumens extending between the proximal portion 18 and the distal portion 16. The elongated shaft 12 may be configured to have a substantially circular cross-section; however, it may be configured to have other suitable cross-sectional shapes, such as elliptical, oval, polygonal, irregular, etc. In addition, the elongated shaft 12 may be flexible along its entire length, or adapted for flexure only along portions of its length. The required degree of flexibility of the elongated shaft 12 may be predetermined based on its intended navigation to a target vascular passage, and the amount of inertial force required for advancing the elongated shaft 12 through the vascular passage. The catheter 10 may be configured as an over-the-wire (OTW) catheter, a single-operator exchange (SOE) catheter, a fixed wire catheter, and/or the like.

The cross-sectional dimensions of the elongated shaft 12 may vary according to the desired application. Generally, the cross-sectional dimensions of the elongated shaft 12 may be sized smaller than the typical blood vessel in which the catheter 10 is to be used. The length of the elongated shaft 12 may vary according to the location of the vascular passage where drug delivery is desired. In some instances, a 6F or a 5F catheter may be used as the elongated shaft 12, where “F,” also known as French catheter scale, is a unit to measure catheter diameter (1F=⅓ mm). In addition, the elongated shaft 12 or a portion thereof may be selectively steerable. Mechanisms such as, pull wires and/or other actuators may be used to selectively steer the elongated shaft 12, if desired.

The proximal portion 18 of the elongated shaft 12 may include a handle 20 usable to manually maneuver the distal portion 16 of the elongated shaft 12. The handle 20 may include one or more ports that may be used to introduce any suitable medical device, fluid or other interventions. For example, the handle 20 (“Y” manifold) may include a guidewire port in conjunction with a guidewire lumen 22 which may be used to introduce a guidewire having an appropriate thickness into the elongated shaft 12, which may guide the shaft 12 to the target location (e.g., target site) within an artery or other vessel. Furthermore, the handle 20 may include an inflation port configured to be coupled to a source of inflation fluid for delivering an inflation fluid through an inflation lumen of the catheter shaft 12 to the inflatable balloon 14. In certain embodiments, the elongated shaft 12 may include one or more additional lumens, which may be configured for a variety of purposes, such as delivering medical devices or for providing fluids, such as saline, to a target location. For instance, the catheter shaft 12 can include an inner lumen (e.g., in a monorail design extending only to the middle segment of the catheter or over-the-wire design extending between the proximal end region and the distal end region) such as an inner guidewire lumen (not illustrated).

The inflatable balloon 14 may be operably coupled at or to the distal portion 16 of the elongated shaft 12. In particular, a proximal portion or waist 24 of the inflatable balloon 14 may be secured to the distal portion 16 of the elongated shaft 12, such as an outer tubular member 26 of the elongated shaft 12. Furthermore, a distal portion or waist 28 of the inflatable balloon 14 may be secured to the distal end region or distal portion 16 of the elongated shaft 12, such as an inner tubular member 30 of the elongate shaft 12 extending through the outer tubular member 26. A suitable securing method(s) may be employed to couple the two structures, including but not limited to adhesive bonding, thermal bonding (e.g., hot jaws, laser welding, etc.) or other bonding technique, as desired. The inflatable balloon 14 may be configured to be expanded from a collapsed (e.g., deflated) configuration to an expanded configuration through delivery of an inflation fluid (e.g., saline) through the inflation lumen of the catheter shaft 12. The balloon 14 may be collapsed during introduction of the catheter inside the patient's body, whereas the balloon 14 may be expanded once it reaches the target site within the body vessel.

Unlike typical drug-delivery balloons which have a substantially circular or cylindrical cross-section, the inflatable balloon 14 may have a non-cylindrical or non-circular cross-section (e.g., taken along a plane that is normal to a longitudinal axis of the catheter 10). For instance, the inflatable balloon 14 can have a symmetric non-circular cross-section such as a quatrefoil formed by four projections 15-1, 15-2, 15-3, 15-4, as shown in the illustrative embodiment in FIGS. 2A-2B. However, in other embodiments the inflatable balloon 14 may have another suitable non-cylindrical configuration or shape (e.g., flat configuration). The inflatable balloon 14 may be manufactured using or otherwise formed of any suitable material, including polymer materials, such as polyamide, polyether block amide (PEBA), polyester, nylon, etc.

The inflatable balloon 14 may include a surface 11 (e.g., a balloon wall or exterior surface) with a therapeutic coating or drug coating 77 (e.g., represented as dots or wavy lines) disposed thereon. In some cases, the therapeutic coating 77 may include encapsulated crystalline everolimus as disclosed herein, for example that is encapsulated with EC, ATBS, or a mixture of EC and ATBC. The therapeutic coating 77 may be disposed along substantially the entire length of the balloon 14 or along one or more regions of the balloon 14. For example, the therapeutic coating 77 may be disposed along a central or body portion of the balloon 14. The therapeutic coating 77 disposed on the balloon 14 (e.g., disposed on a surface 11 or balloon wall of the balloon 14) may have an average thickness in the range of about 1 micron (0.0000394 inches) to about 50 microns (0.00197 inches), for example.

The surface 11 can include a first region (e.g., first portion) and a second region (e.g., second portion). The first region and the second region refer to distinct regions on a surface of a balloon. For instance, each projection 15-1, 15-2, 15-4, and 15-4 can include a respective first region and a respective second region forming at least a portion of a surface of each projection. In some embodiments, the first region and the second region refer to distinct non-overlapping regions on the surface 11 of the balloon 14.

The first region can be uncoated (e.g., has an absence of the therapeutic coating 77). The second region can be coated (e.g., the therapeutic coating 77 is present). Having the first region be uncoated can promote aspects herein such as minimizing frictional loss of the therapeutic coating (e.g., which is only applied to the second region of the balloon 14). That is, in contrast to other approaches such as those that employ cylindrical or spherical balloons having an entire exterior surface (e.g., an entire abluminal surface) coated with a therapeutic coating and therefore may be prone to loss of at least a portion of the coating that contacts tissue of a vessel during navigation of the balloon to a target site, the balloons herein can include uncoated first regions that may contact tissue during navigation of the balloon to the target site, and also include coated second regions that may be configured to avoid (not contact) tissue during navigation of the balloon to the target site. It also may be possible to have a covering sheath or sleeve over the coated balloon that can be retracted before balloon inflation.

In some embodiments, the balloons herein can include a plurality of first regions and/or a plurality of second regions. For instance, the balloon herein can include a plurality of first regions and a plurality of second regions. In some embodiments, a quantity of first region(s) can be equal to a quantity of second region(s). For instance, the surface can include an individual (e.g., only one) first region and an individual second region, can include two first regions and two second regions, can include three first regions and three second regions, can include four first regions and four second regions, and/or can include five first regions and five second regions, among other possibilities. Having a quantity of the first regions be equal to a quantity of the second regions can promote aspects herein such as promoting formation of uniform perfusion channels about the balloon 14 when the balloon 14 is expanded. However, in some embodiments a quantity of the first regions can be different than a quantity of the second regions.

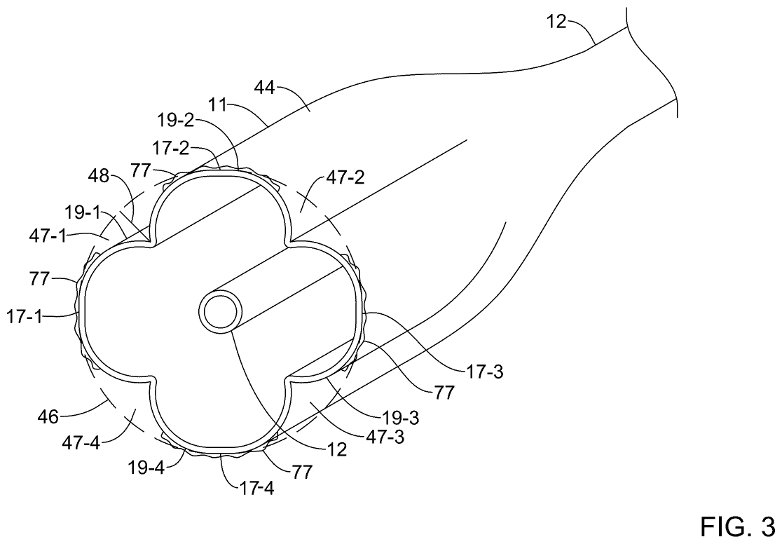

In some embodiments, the first region, the second region, or both the first region and the second region can move (e.g., radially relative to a longitudinal axis of the perfusion catheter 10) between a first (unexpanded) configuration of a balloon and a second (expanded) configuration of the balloon. For instance, the first region, the second region, or both can be formed of materials (e.g., shape memory materials) and/or can have different thickness and/or types of materials at different portions thereof to cause the regions to move between the collapsed and expanded configurations. In some embodiments, each of the first regions, each of the second regions, or both each of the first regions and each of the second regions can move (e.g., radially) between a first (unexpanded) configuration of a balloon and a second (expanded) configuration of the balloon. For instance, each of the second regions 17-1, 17-2, 17-3, and 17-4 can move (e.g., radially) relative to each of the first regions 19-1, 19-2, 19-3, and 19-4 between the first configuration and the second configuration. For instance, the first regions or the second regions can be fixed (e.g., remain substantially the same distance from the tissue of the vessel wall 46 (shown in FIG. 3) and/or the longitudinal axis (LA) of the perfusion catheter 10) in one or both of the first (collapsed) configuration and the second (expanded) configuration. For example, the first regions can be fixed (e.g., remain substantially the same distance from the tissue and/or the longitudinal axis of the perfusion catheter 10) in both the first (collapsed) configuration and the second (expanded) configuration, as illustrated in FIGS. 2A-2B. However, the second regions can move (e.g., rotate and/or translate) between the first (collapsed) configuration and the second (expanded) configuration. For example, the second regions can move at least radially between the first (collapsed) configuration and the second (expanded) configuration, as illustrated in FIGS. 2A-2B.

In some embodiments, a second region of a balloon such as balloon 14 can be configured to be a first distance from a longitudinal axis of the catheter 10 in the collapsed configuration, and can be configured to be a second distance from the longitudinal axis of the perfusion catheter 10 in the expanded configuration, where the second distance is greater than the first distance. Stated differently, the second region of the balloon can be configured to be a first distance from the tissue of the vessel wall 46 when the balloon is in a first (collapsed) configuration and the second region can be configured to be a second distance from the tissue that is less than the first distance from the tissue of the vessel wall 46. For instance, the first distance from the tissue of the vessel wall 46 can be a non-zero number and the second distance from the tissue can be equal to zero (e.g., the second region is configured to directly contact the tissue of the vessel wall 46) when the balloon is in the second (expanded) configuration. However, in some embodiments the first regions 19-1, 19-2, 19-3, and 19-4 can move (e.g., radially) relative to the second regions 17-1, 17-2, 17-3, and 17-4 between the first configuration and the second configuration, as described herein with respect to FIGS. 2A-2B.

In some embodiments, the first region can be included in a plurality of first regi ons and the second region can be included in a plurality of second regions. In some embodiments, the plurality of first regions and the plurality of second regions can alternate (e.g., be disposed in alternating fashion about a circumference or exterior surface of the balloon 14). Stated differently, each of the plurality of first regions and each of the plurality of second regions can together form alternating regions that alternate about an exterior (abluminal) surface of the balloon 14. For instance, as illustrated in FIGS. 2A-2B and FIG. 3, the surface of the balloon 14 can include a plurality of first regions 19-1, 19-2, 19-3, and 19-4 that alternate with a plurality of second regions 17-1, 17-2, 17-3, and 17-4. That is, the respective first regions of the plurality of first regions 19-1, 19-2, 19-3, and 19-4 can alternate with respective second regions of the plurality of second regions 17-1, 17-2, 17-3, and 17-4. Having the plurality of first regions alternate with the plurality of second regions can promote aspects herein such as promoting the formation of perfusion channels 47-1, 47-2, 47-3, and 47-4 and/or protecting (e.g., by shielding the coated second regions with the uncoated first regions) the therapeutic coating on the balloon 14 during insertion and/or navigation of the balloon 14 to a target site.

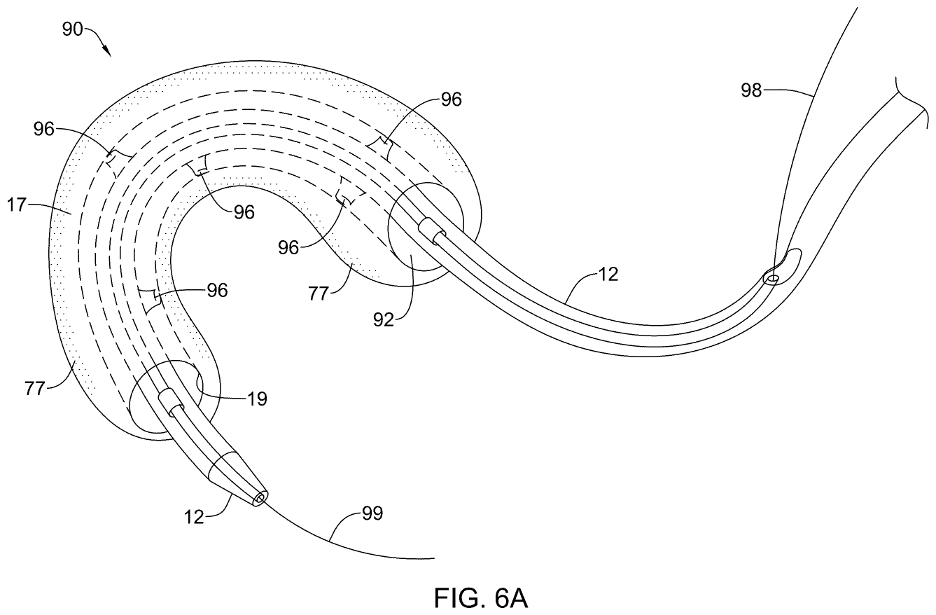

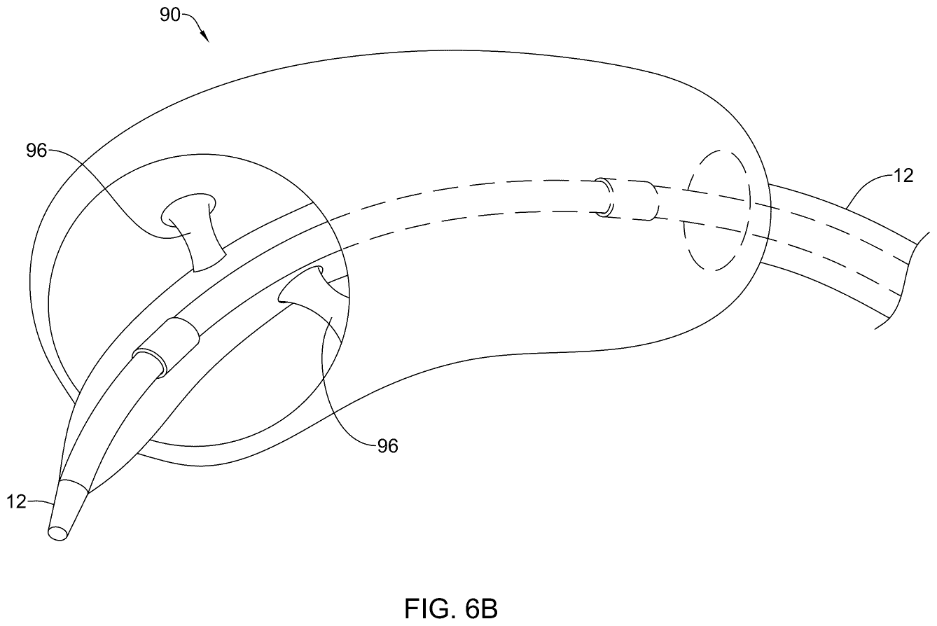

In some embodiments, the perfusion channels can be formed when the balloons herein are in an expanded configuration. In some embodiments, the perfusion channels may be absent (not formed) when the balloons herein are in an unexpanded configuration. The perfusion channels can be substantially longitudinally extending perfusion channels that are configured to permit perfusion of blood from a first side of the balloon to a second (e.g., opposing) side of the balloon when the balloon is in the expanded configuration. For instance, the perfusion channels herein can be continuous perfusion channels that extend in an uninterrupted manner between a distal end to a proximal end of a perfusion balloon. Having the perfusion channels be continuous perfusion channels that extend in an uninterrupted manner (e.g., do not include structures within the volume of the perfusion channels) can promote aspects herein such as permitting blood to readily flow through the perfusion channels when the balloon is in an expanded configuration. However, in some embodiments such as those which employ an individual perfusion channel a plurality of struts (e.g., struts 96 as described with respect to FIGS. 6A-6B) can be present within a volume of the individual perfusion channel.

In some embodiments, the perfusion channel can be an individual perfusion channel, for instance as illustrated in FIGS. 6A-6B. However, in some embodiments the perfusion channels can be formed of a plurality of respective perfusion channels. In any case, the perfusion channel can be configured to permit a sufficient amount of blood to flow about the balloon when the balloon is in a second (expanded) configuration and thereby maintain sufficient perfusion of tissue (e.g., cardiac tissue) that is distal to the expanded balloon. For instance, the perfusion channels can be configured with a width in a range from about 2 microns (0.0000787 inches) to about 50 microns (0.00197 inches) and a height in a range from about 2 (0.0000787 inches) microns to about 50 microns (0.00197 inches). All individual values and sub-ranges between about 2 microns (0.0000787 inches) to about 50 microns (0.00197 inches) are included. Having the perfusion channels be configured with a width in a range from about 2 microns (0.0000787 inches) to about 50 microns (0.00197 inches) and a height in a range from about 2 microns (0.0000787 inches) to about 50 microns (0.00197 inches) can ensure that a sufficient amount of blood flows through the perfusion channels when the balloon is an a second (expanded) configuration.

Coronary blood flow is subject to a wide variation, depending on the heart's activity: from 70 to 80 milliliters (mL)/minute (min) for 100 g tissue at rest, to as much as 300-400 mL/min per 100 g tissue on exertion. The resting coronary blood flow is ˜250 ml per min. The flow rate of blood though the perfusion channels about the balloon when in the second (expanded) configuration can be greater than about 20 milliliters per minute, greater than about 30 milliliters per minute, greater than about 40 milliliters per minute, and/or greater than about 50 milliliters per minute. In some embodiments, the flow rate of blood through the perfusion channels about the balloon when in the expanded configuration can be in a range from about 20 to about 60 milliliters per minute, about 20 to about 50 milliliters per minute, about 20 to about 40 milliliters per minute, or about 20 to about 30 milliliters per minute. All individual values and sub-ranges from about 20 to about 50 milliliters per minute are included. For instance, a flow of the blood through each of the perfusion channel can be greater than about 20 milliliters per minute thereby ensuring sufficient perfusion of tissue (e.g., cardiac tissue) proximal to the balloon when the balloon is expanded. For example, a sum of respective flow rates of the blood through each of a plurality of perfusion channels of a given balloon can be greater than about 20 milliliters per minute to ensure sufficient perfusion to a target site and/or to a portion of a vessel that is distal to the target site (e.g., to maintain perfusion to cardiac tissue distal to the expanded balloon).

In some embodiments, the perfusion channels can be formed of respective gaps between a first region of the surface of a balloon and tissue (e.g., a vessel wall) adjacent to the balloon. For instance, as illustrated in FIG. 3 the perfusion channels 47-1, 47-2, 47-3, and 47-4 can be formed of respective gaps (e.g., annular gaps) between a respective first regions 19-1, 19-2, 19-3, 19-4 of the surface 11 of the balloon 14. That is, when the balloon 14 is in an expanded configuration the first regions 19-1, 19-2, 19-3, and 19-4 can be spaced a distance (e.g., distance 48) from the tissue of the vessel wall 46 in which the balloon 14 is disposed. In some embodiments, respective portions (but not all) of the surface area of the first regions herein are spaced (e.g., radially) a distance from the tissue of the vessel wall 46. However, in some embodiments the entire surface area of the first regions 19-1, 19-2, 19-3, and 19-4 can be spaced (e.g., radially) a distance away from the tissue of the vessel wall 46.

FIG. 3 depicts an example drug-coated balloon in an expanded configuration in contact with a vessel wall showing the perfusion gaps to allow for fluid (i.e., blood) passage during balloon inflation. As illustrated in FIG. 3, the perfusion channels 47-1, 47-2, 47-3, and 47-4 can be substantially the same shape and substantially the same size (e.g., having the same volume). Having the perfusion channels herein be substantially the same shape and sustainably the same size can promote aspects herein such as promoting efficient and uniform blood flow about the balloons herein when the balloons are in an expanded configuration. However, in some instances the perfusion channels can be configured with different shapes and/or different sizes.

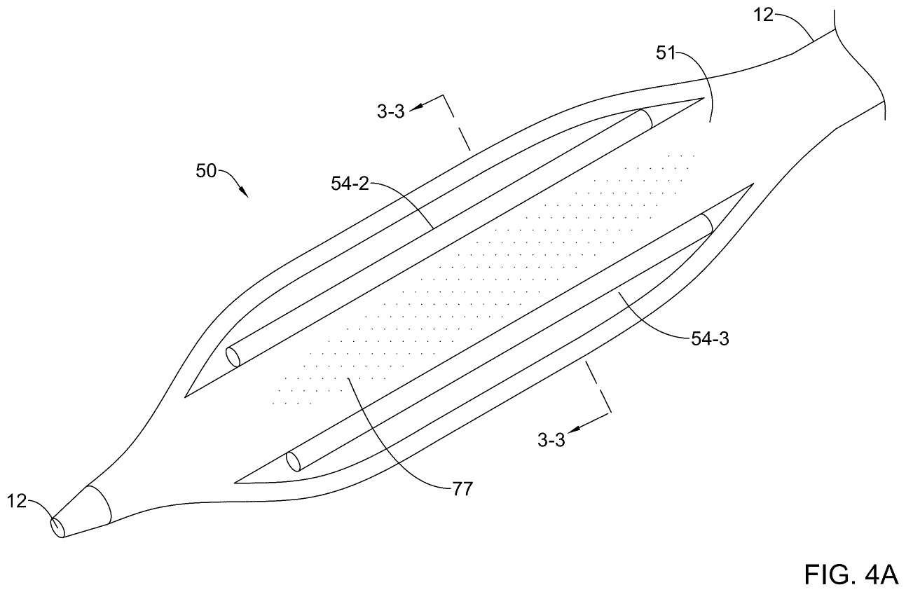

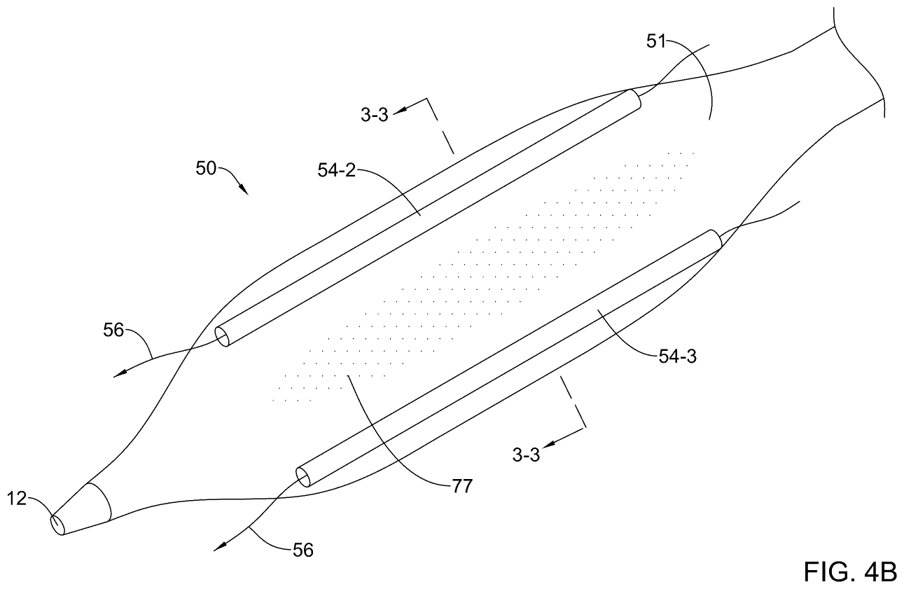

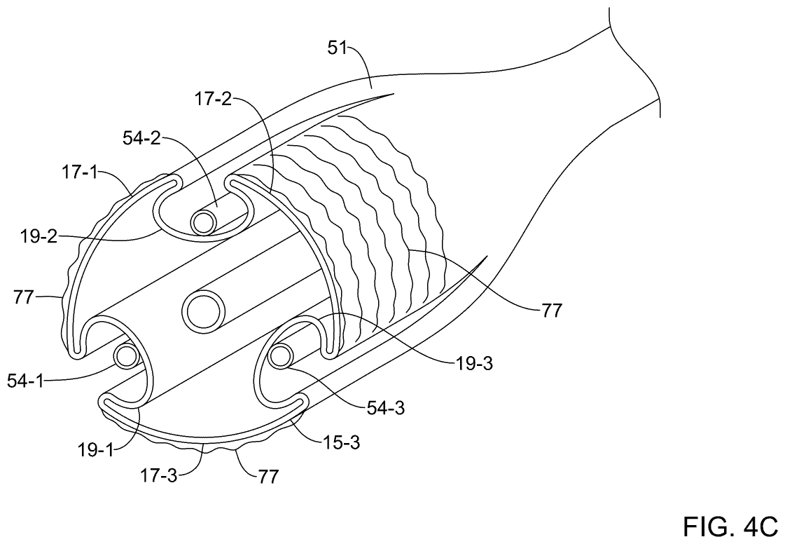

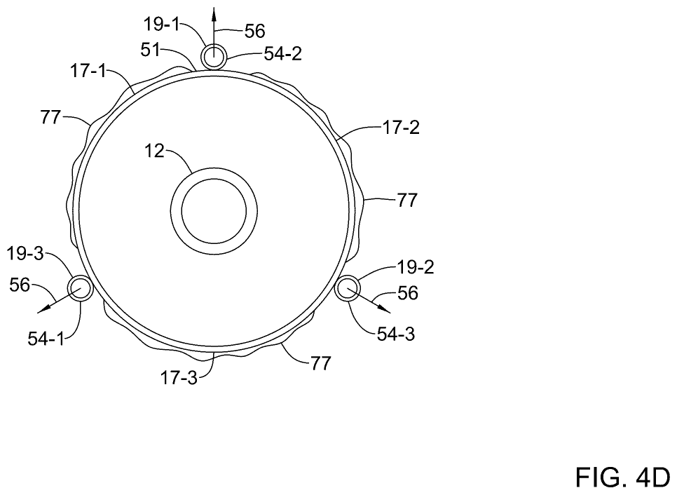

While FIG. 3 illustrates four perfusion channels 47-1, 47-2, 47-3, and 47-4, the quantity of perfusion channels can be increased or decreased. For instance, the perfusion channel can be formed of an individual perfusion channel, as illustrated in FIGS. 6A-6B. Additionally, while FIG. 3 illustrates the perfusion channels 47-1, 47-2, 47-3, and 47-4 as being formed of gaps between the respective first regions 19-1, 19-2, 19-3, and 19-4 and the tissue of the vessel wall 46, other configurations are possible. For instance, the perfusion channels can be an individual channel manifested as a lumen extending longitudinally through a toroidal or other shaped balloon, as illustrated in FIGS. 6A-6B, or can be formed at least in part of a tubular member, as illustrated in FIGS. 4A-4D, among other possibilities.

FIGS. 4A-4B illustrate another example drug-coated balloon 50 in a collapsed or delivery configuration (FIG. 4A) and in an expanded or deployed configuration (FIG. 4B). FIG. 4A illustrates another example drug-coated balloon in a collapsed configuration with tubular structures to allow for fluid (i.e., blood) passage during balloon inflation. FIG. 4B illustrates the balloon of FIG. 4B in an expanded configuration with the tubular structures allowing for fluid (i.e., blood) passage during balloon inflation. FIGS. 4C-4D illustrate cross-sections (taken at section line 3-3) of the drug-coated balloon 50 in a collapsed or delivery configuration (FIG. 4C) and in an expanded or deployed configuration (FIG. 4D). In general, the balloons herein may be delivered to a suitable target site (in a vessel) via a catheter/delivery system while in the collapsed configuration. Upon reaching the target site, the balloons may expand or be expanded into the expanded configuration. For instance, the balloon may be constrained crimped or folded into a delivery device/catheter and then expanded (e.g., via an expandable member or balloon) when at/adjacent the target site.

Similar to the balloon 14, the balloon 50 is a component of a drug delivery balloon catheter (e.g., drug delivery balloon catheter 10 as illustrated in FIG. 1) that includes an elongated shaft 12 with the inflatable drug-delivery balloon 50 coupled at or to a distal portion of the shaft 12. The balloon 50 has a surface 51 comprised of plurality of first (uncoated) regions 19-1, 19-2, and 19-3 and a plurality of second (coated) regions 17-1, 17-2, and 17-3, which may be similar to the first regions and second regions described with respect to the balloon 14 in FIGS. 2A-2B and FIG. 3. For instance, as illustrated in FIGS. 4A-4D, the first regions 19-1, 19-2, and 19-3 can correspond to uncoated regions of the surface 51 that alternate with the second regions 17-1, 17-2, and 17-3 which correspond to coated regions of the surface 51.

The balloons herein can in some embodiments include an elongate perfusion tube. For instance, the balloon 50 can include a plurality of elongate perfusion tubes 54-1, 54-2, and 54-3, as illustrated in FIGS. 4A-4D. The elongate perfusion tubes can be coupled to, disposed on, or at least partially embedded within the surface 51. The elongate perfusion tubes can extend substantially between a distal end and a proximal end of the balloon 50, as illustrated in FIGS. 4A-4B. The elongate perfusion tubes can be hollow substantially longitudinally extending tubular members that are configured to permit blood to flow substantially longitudinally about the balloon 50 when the balloon 50 is in a second (expanded) configuration. For instance, the elongate perfusion tubes 54-1, 54-2, and 54-3 may together permit flow rate of the blood (represented as element 56 in FIGS. 4B and 4D) that is greater than about 20 milliliters per minute to ensure that sufficient perfusion about the balloon 50 is maintained. For instance, each of the elongate perfusion tubes may be hollow tubes with an uninterrupted longitudinally extending lumen extending therethrough (extending from a distal end to a proximal end of each respective elongate perfusion tube). That is, in some embodiments the perfusion channel can be formed at least in part by a lumen of an elongate perfusion tube that is coupled to and extending longitudinally along the surface 51 of the balloon 50. In some embodiments, the perfusions channels herein can be manifested entirely as a plurality of elongate perfusion tubes such as the elongate perfusion tubes 54-1, 54-2, and 54-3.

When the balloon 50 is in a first (collapsed) configuration the first regions 19-1, 19-2, and 19-3 can each be recessed (axially) a distance with respect to the second regions 17-1, 17-2, and 17-3. Having first regions 19-1, 19-2, and 19-3 be recessed (axially) a distance with respect to the second regions 17-1, 17-2, and 17-3 can thereby recess the plurality of elongate perfusion tubes a distance (axially) with respect to the second regions. As such, the elongate perfusion tubes may be protected by the second regions during insertion or delivery of the balloon to a target site. However, when the balloon 50 is in a second (expanded) configuration the first regions 19-1, 19-2, and 19-3 can protrude (axially) a distance with respect to the second regions 17-1, 17-2, and 17-3. Having first regions 19-1, 19-2, and 19-3 protrude (axially) a distance with respect to the second regions 17-1, 17-2, and 17-3 when the balloon is in the second (expanded) configuration can ensure that sufficient blood flow is maintained (e.g., blood flows through the lumens of the perfusion tubes) and may also help retain the balloon at a target site thereby enhancing drug delivery from the therapeutic coating on the plurality of second regions of the balloon 50.

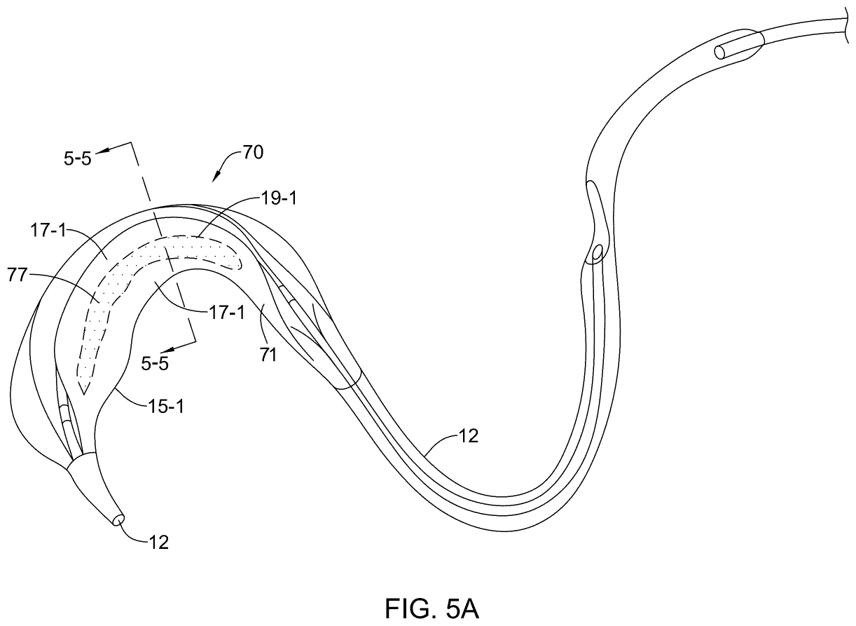

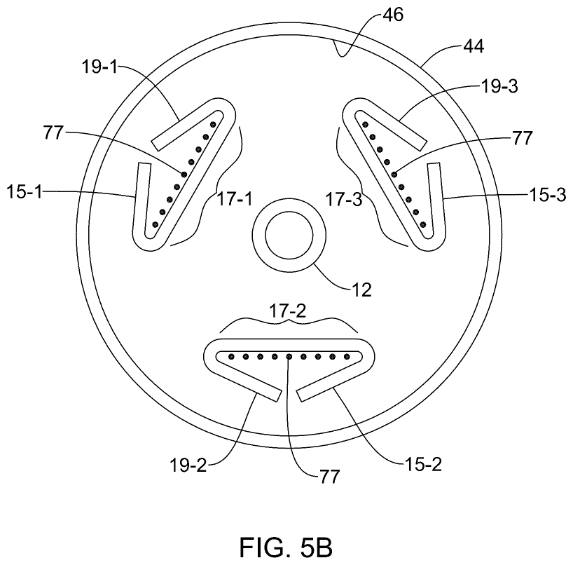

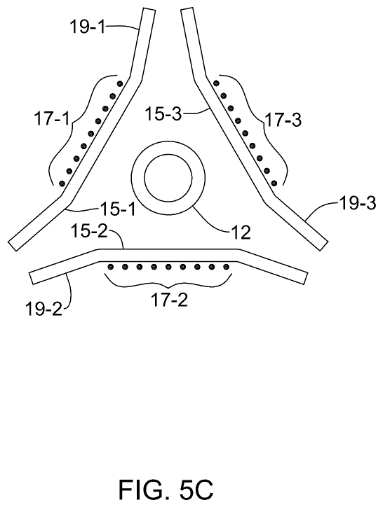

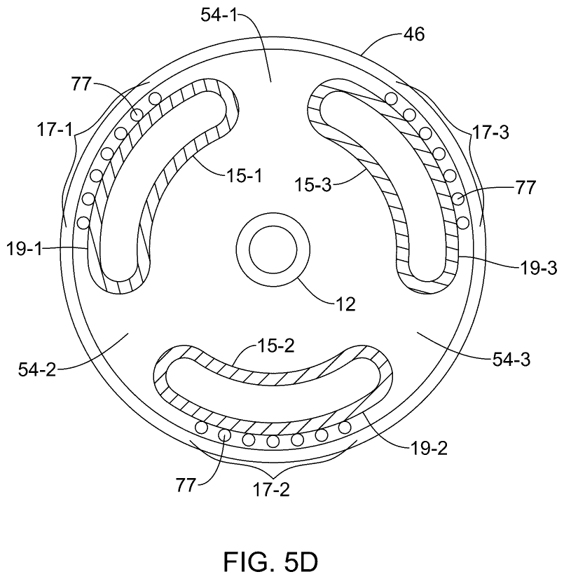

FIG. 5A and FIG. 5B illustrate an example drug-coated balloon 70 and a cross-sectional view of the balloon 70 (taken along the Line 5-5) in a collapsed configuration. FIG. 5A illustrates an example drug delivery balloon monorail catheter (guidewire exit on middle segment) with a multi-lobular (three lobes) drug-coated balloon. FIG. 5B illustrates a cross-sectional view taken through line 5-5 of the multi-lobular (three lobes) balloon of FIG. 5A in a collapsed configuration, with the drug reservoir occluded to avoid drug losses when tracking the catheter through vasculature. Similar to the balloons 14 and 50, the balloon 70 is a component of a drug delivery balloon catheter (e.g., drug delivery balloon catheter 10 as illustrated in FIG. 1) that includes an elongated shaft 12 with the inflatable drug-delivery balloon 70 coupled at or to a distal portion of the shaft 12. The balloon 70 has a surface 71 comprised of plurality of first (uncoated) regions 19-1, 19-2, and 19-3 and a plurality of second (coated) regions 17-1, 17-2, and 17-3, which may be similar to the first regions and second regions described with respect to the balloon 14 in FIGS. 2A-2B and FIG. 3 and/or balloon 50 in FIGS. 4A-4D. For instance, as illustrated in FIGS. 5A-5D, the first regions 19-1, 19-2, and 19-3 can correspond to uncoated regions of the surface 71 that alternate with the second regions 17-1, 17-2, and 17-3 which correspond to coated regions of the surface 71.

As illustrated in FIGS. 5A-5D, the balloon 70 can include a plurality of projections 15-1, 15-2, and 15-3 that extend in a substantially longitudinal direction. Each of the projections 15-1, 15-2, and 15-3 can be configured to move between a first (unexpanded) configuration and a second (expanded) configuration. For instance, each of the projections 15-1, 15-2, and 15-3 can be in a first (unexpanded) configuration, as illustrated in FIG. 5B. When in the first configuration, each of the projections 15-1, 15-2, and 15-3 can be substantially closed such that the first regions 19-1, 19-2, and 19-3 at least partially overlay (e.g., are positioned radially above) the corresponding second regions 17-1, 17-2, and 17-3, as illustrated in FIG. 5B. That is, FIG. 5B illustrates a cross-sectional view of the balloon of FIG. 5A in a collapsed or delivery configuration. Having each of the projections 15-1, 15-2, and 15-3 be substantially closed when the balloon 70 is in the first configuration can promote aspects herein such as protecting a therapeutic coating 77 disposed on an exterior surface of the second regions 17-1, 17-2, and 17-3 while the balloon 70 is navigated or delivered to a target site. For instance, the first regions 19-1, 19-2, and 19-3 may contact tissue while the balloon 70 is navigated or delivered to a target site, but the therapeutic coating 77 on the surface of the second regions 17-1, 17-2, and 17-3 may be not contact the tissue while the balloon 70 is navigated or delivered to a target site. Thus, when in the closed configuration or delivery configuration, each of the second regions 17-1, 17-2, and 17-3 may remain spaced a distance from tissue of a vessel and may be at least partially overlaid with a corresponding first region 19-1, 19-2, and 19-3. As such, the approaches herein can ensure that an intended amount of therapeutic coating is delivered to and available for drug delivery at the target site as compared to other typical approaches that may be prone to frictional loss of at least a portion of a therapeutic coating during navigation or delivery of a drug-coated balloon to a target site.

FIG. 5C illustrates a cross-sectional view taken through line 5-5 of the multi-lobular (three lobes) balloon of FIG. 5A in a partially expanded configuration exposing the drug load reservoir. The balloon 70 can transition from the closed configuration in FIG. 5A to the partially expanded or partially deployed configuration in FIG. 5C when the balloon 70 is located at the target site (e.g., a target site adjacent to tissue in a vessel). For instance, the balloon 70 can begin to undergo expansion such that each of the projections 15-1, 15-2, and 15-3 moves relative to a longitudinal axis of the balloon 70. For example, each of the first regions 19-1, 19-2, and 19-3 can rotate (e.g., due to difference in material thickness, difference in material type, and/or the presence of shape memory material in the balloon 70) about the respective second regions 17-1, 17-2 and 17-3 to an open configuration (e.g., a partially expanded configuration), as shown in FIG. 5C. The rotation can occur responsive to the introduction of fluid via an inflation lumen in the shaft 12 to the balloon 70. When in the open configuration or partially deployed configuration, each of the first regions 19-1, 19-2, and 19-3 and the second regions 17-1, 17-2, and 17-3 can together form concave protrusions 15-1, 15-2, and 15-3 (e.g., concave with respect to the longitudinal axis of the catheter 10), as shown in FIG. 5C. When in the open configuration or partially deployed configuration, each of the second regions 17-1, 17-2, and 17-3 of the balloon 70 may remain spaced a distance from tissue of a vessel at a target site.

FIG. 5D illustrates a cross-sectional view taken through line 5-5 of the multi-lobular (three lobes) balloon of FIG. 5A in an expanded configuration with the drug reservoir exposed and apposed to the vessel wall for drug transfer. The balloon 70 can transition from the partially expanded configuration illustrated in FIG. 5C to the expanded or deployed configuration in FIG. 5D when the balloon 70 is located at the target site. For instance, the balloon 70 can continue to undergo expansion from the partially expanded state such that each of the projections 15-1, 15-2, and 15-3 moves relative to a longitudinal axis of the balloon 70. For example, each of the second regions 17-1, 17-2, and 17-3 can move (radially) relative to the respective first regions 19-1, 19-2 and 19-3 to an expanded configuration, as shown in FIG. 5D. When in the expanded configuration, each of the first regions 19-1, 19-2, and 19-3 and the second regions 17-1, 17-2, and 17-3 can together form convex protrusions 15-1, 15-2, and 15-3, as shown in FIG. 5D. When in the expanded configuration (fully deployed configuration), each of the second regions 17-1, 17-2, and 17-3 may be the most proximate surface of the projections 15, 15-2, and 15-3 to tissue of a vessel at a target site. For instance, when in the expanded configuration or deployed configuration, each of the second regions 17-1, 17-2, and 17-3 may contact tissue of a vessel at a target site. Thus, the therapeutic coating 77 disposed on the second regions 17-1, 17-2, and 17-3 may contact the tissue of the vessel wall 46 when the balloon 70 is in the expanded configuration, as illustrated in FIG. 5D. In some embodiments, when the balloon 70 is in the expanded configuration, each of the second regions 17-1, 17-2, and 17-3 may contact tissue of a vessel at a target site and each of the first regions 19-1, 19-2, and 19-3 may be spaced a distance away from (not contact) the tissue at the target site. As such, a plurality of perfusion channels 54-1, 54-2, and 54-3 can be formed between opposing first regions of adjacent projections 15-1, 15-2, and 15-3, as illustrated in FIG. 5D.

As detailed above, movement of the first regions and second regions of the balloon 70 between the first (unexpanded) configuration (FIG. 5B), the partially deployed configuration (FIG. 5C), and the second (expanded) configuration (FIG. 5D) can cause the first region, the second region, or both the first region and the second region to be different distances from the tissue 44 depending on a configuration of the balloon 70. For instance, the first regions 19-1, 19-2, and 19-3 can remain a distance away from the vessel wall 46 when in the balloon 70 is in the first configuration, the partially deployed configuration, and the deployed configuration. However, the second regions 17-1, 17-2, and 17-3 can be positioned a distance away from the vessel wall 46 when the balloon 70 is the first configuration and the partially expanded configuration, and can be positioned to contact the vessel wall 46 when the balloon 70 is in the expanded configuration.

FIG. 6A illustrates an example drug-coated toroidal balloon 90 in an expanded or deployed configuration. FIG. 6B illustrates another view of the toroidal balloon of FIG. 6A. The balloon 90 can include a guidewire 99 extending therethrough (e.g., extending through the shaft 12). The balloon 90 can be a toroidal shaped balloon, as illustrated in FIGS. 6A-6B. The balloon 90 can be a toroidal shaped balloon having a lumen 92 extending substantially longitudinally therethrough. In such instances, a perfusion channel can be an individual perfusion channel manifested as the lumen 92. Thus, the lumen 92 can be configured to permit blood to flow substantially longitudinally about the balloon 90 (e.g., from a distal end of the balloon to a distal end of the balloon 90). For instance, the lumen 92 can be configured such that the flow of blood therethrough is greater than about 20 milliliters per minute when the balloon 90 is in an expanded configuration. The patency of the lumen 92 can be maintained at least in part due to the presence of a plurality of struts 96 which are positioned longitudinally along and circumferentially about the shaft 12. That is, the struts 96 can be hollow or include a lumen that fluidically couples an inflation lumen 98 in the shaft 12 to an interior volume within a surface of the balloon 90. Thus, fluid may be conveyed inside of the surface of the balloon 90 via the shaft 12 and struts 96 to cause the balloon 90 to transition to or remain in a second (expanded) configuration. As illustrated in FIGS. 6A and 6B, the struts 96 may project substantially axially from the shaft 12 and be coupled to a luminal surface of the balloon 90. For instance, a first strut may project in a first direction from the shaft 12 and a second strut that is adjacent to (distal or proximal to) the first strut may project from the shaft 12 in a second direction that is substantially opposite the first direction. In such embodiments, a third strut that is adjacent to the second strut may project in substantially the first direction. That is, adjacent struts extending longitudinally along the shaft may be configured in alternative direction (e.g., substantially opposite directions), as illustrated in FIGS. 6A-6B. Having longitudinally adjacent struts be configured in alternative (opposing) directions can promote aspects herein such as maintaining the patency of the lumen 92 when the balloon 90 is in the second (expanded) configuration. In the embodiments shown in FIGS. 6A-6B, the therapeutic coating 77 can be disposed on some or all of a second region 17 (e.g., some or all of the abluminal surface) of the balloon 90. For instance, the therapeutic coating 77 can be disposed on substantially all of the second region 17 and can be absent from a first region 19 (e.g., a luminal surface) of the balloon 90, as illustrated in FIGS. 6A-6B. In the embodiments shown in FIGS. 6A-6B, the balloon 90 can include an individual first region 19 and an individual second region 17. However, in various embodiments the balloons herein can include a plurality of first regions and/or a plurality of second regions, as described herein.

The materials that can be used for the various components of the medical devices balance different degrees of compromise between flexibility and stiffness which are required to navigate the relevant segments of the human vasculature. More rigid shafts can be used in the proximal segment of a catheter (e.g., metallic hypotubes) to increase the push of the catheter. More flexible shafts can be used in the distal segment of a catheter to gain access to tortuous anatomy. An inflatable balloon can be designed for different degrees of compliance during balloon pressurization (non-compliant, semi-compliant, fully compliant) by using co-polymers alternating rigid blocks and flexible blocks. The materials described herein may include those commonly associated with medical devices. The medical devices described herein may include components that may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N 06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R 30035 such as M P 35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R 30003 such as ELGILOY®, PHY NOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the medical devices described herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical devices described herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical devices described herein to achieve the same result.

In some embodiments, a degree of Magnetic Resonance Imaging (MRI) compatibility is imparted into the medical devices described herein. For example, the medical devices described herein, or portions thereof, may be made of a material that does not substantially distort the image and create substantial artifacts (i.e., gaps in the image). Certain ferromagnetic materials, for example, may not be suitable because they may create artifacts in an MRI image. The medical devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHY NOX®, and the like), nickel-cobalt-chromium-molybdenum alloys (e.g., UNS: R 30035 such as M P 35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A perfusion catheter for drug delivery to tissue, the perfusion catheter comprising:
an elongate catheter shaft having a proximal end region and a distal end region and including an inflation lumen extending between the proximal end region and the distal end region;
a balloon positioned adjacent to the distal end region of the elongate catheter shaft and in fluid communication with the inflation lumen, the balloon being configured to move between a collapsed configuration and an expanded configuration where a first region of a surface of the balloon defines a perfusion channel; and
a therapeutic coating disposed on a second region of the surface of the balloon, wherein the second region is configured to contact the tissue in the expanded configuration.

2. The perfusion catheter of claim 1, wherein the first region is free of the therapeutic coating.

3. The perfusion catheter of claim 1, wherein the perfusion channel is configured to permit blood to flow substantially longitudinally about the balloon, and wherein a flow rate of the blood is greater than about 20 milliliters per minute.

4. The perfusion catheter of claim 1, wherein the perfusion channel has a width in a range from about 2 microns (0.0000787 inches) to about 50 microns (0.00197 inches) and a height in a range from about 2 microns (0.0000787 inches) to about 50 microns (0.00197 inches).

5. The perfusion catheter of claim 1, wherein the perfusion channel is formed of a plurality of perfusion channels.

6. The perfusion catheter of claim 5, wherein each of the plurality of perfusion channels is substantially the same shape, substantially the same size, or both.

7. The perfusion catheter of claim 6, wherein the second region is configured to be a first distance from a longitudinal axis of the perfusion catheter in the collapsed configuration, a second distance from the longitudinal axis of the perfusion catheter in the expanded configuration, and wherein the second distance is greater than the first distance.

8. The perfusion catheter of claim 1, wherein the perfusion channel is an individual perfusion channel.

9. The perfusion catheter of claim 8, wherein the balloon is a toroidal shaped balloon, and wherein the individual perfusion channel is a lumen extending through the toroidal shaped balloon.

10. The perfusion catheter of claim 9, further comprising struts extending radially from the elongate catheter shaft and being in fluid communication with the toroidal shaped balloon and an inflation lumen in the elongate catheter shaft.

11. The perfusion catheter of claim 1, wherein the second region is configured to be recessed a distance from the tissue when the balloon is in the collapsed configuration.

12. The perfusion catheter of claim 1, wherein the perfusion channel further comprises a substantially longitudinally extending perfusion channel that is configured to permit perfusion of blood from a first side of the balloon to a second side of the balloon when the balloon is in the expanded configuration.

13. The perfusion catheter of claim 1, wherein the perfusion channel comprises a gap between the first region of the surface of the balloon and the tissue.

14. The perfusion catheter of claim 1, wherein the perfusion channel is formed at least in part by an elongate perfusion tube coupled to and extending longitudinally along the surface of the balloon.

15. The perfusion catheter of claim 1, wherein the therapeutic coating comprises a plurality of everolimus crystals or a plurality of paclitaxel crystals.

16. A perfusion catheter for drug delivery to cardiac tissue, the perfusion catheter comprising:
an elongate catheter shaft having a proximal end region and a distal end region and including a guidewire lumen and an inflation lumen extending between the proximal end region and the distal end region;
a balloon positioned adjacent to the distal end region of the elongate catheter shaft and in fluid communication with the inflation lumen, the balloon being configured to move between a collapsed configuration and an expanded configuration where a first region of an exterior surface of the balloon is uncoated and defines a perfusion channel configured to permit substantially longitudinal blood flow about the balloon; and
a therapeutic coating disposed on a second region of the exterior surface of the balloon, wherein the second region is configured to contact the cardiac tissue in the expanded configuration.

17. The perfusion catheter of claim 16, wherein the first region is included in a plurality of first regions, wherein the second region is included in a plurality of second regions.

18. The perfusion catheter of claim 17, wherein the plurality of first regions and the plurality of second regions are configured to alternate about an abluminal surface of the balloon.

19. A perfusion catheter for drug delivery to cardiac tissue, the perfusion catheter comprising:
an elongate catheter shaft having a proximal end region and a distal end region and including an inflation lumen extending between the proximal end region and the distal end region;
a balloon positioned adjacent to the distal end region of the elongate catheter shaft and in fluid communication with the inflation lumen, the balloon being configured to move between an collapsed configuration and a radially expanded configuration where a plurality of first regions of an exterior surface of the balloon are uncoated and define a plurality of perfusion channels extending substantially longitudinally about the exterior surface of the balloon and being configured to permit blood to flow substantially longitudinally about the exterior surface, wherein a sum of the respective flow rates of the blood through each of the plurality of perfusion channels is greater than about 20 milliliters per minute; and
a therapeutic coating disposed on a plurality of second regions of the exterior surface of the balloon, wherein the plurality of second regions are configured to contact the cardiac tissue in the radially expanded configuration.

20. The perfusion catheter of claim 19, wherein:
the plurality of first regions each have a substantially concave shape relative to a longitudinal axis of the perfusion catheter when the balloon is in the collapsed configuration; and
the plurality of second regions each have a substantially convex shape relative to the longitudinal axis of the perfusion catheter when the balloon is in the radially expanded configuration.
